(19) United States
(12) Patent Application Publication
(10) Pub. No.: US 2014/0058255 A1
(43) Pub. Date: Feb. 27, 2014

(54) Physiological Information Detection System

(71) Applicant: Kyushu TLO Company, Limited, Fukuoka (JP)

(72) Inventors: Atsushi Mase, Fukuoka (JP); Yutaka Komada, Fukuoka (JP); Naoki Ito, Fukuoka (JP); Eiji Sakata, Fukuoka (JP); Hiroshi Shimazu, Fukuoka (JP)

(73) Assignee: Kyushu TLO Company, Limited, Fukuoka (JP)

(21) Appl. No.: 13/974,943

(22) Filed: Aug. 23, 2013

Related U.S. Application Data
(63) Continuation-in-part of application No. PCT/JP2012/054537, filed on Feb. 24, 2012.

(30) Foreign Application Priority Data
Feb. 25, 2011 (JP) 2011-039954

(51) Int. Cl. A61B 5/05

(52) U.S. Cl. CPC A61B 5/0507
USPC 600/430

(57) ABSTRACT

A living organism information detection system is capable of appropriately acquiring desired information such as the existence of a living organism, the condition of the living organism, or the like by irradiating a region to be measured with an electromagnetic wave while scanning the region to be measured to thereby cause the transmission/reception of the electromagnetic wave to/from the living organism within the region to be measured to be appropriately performed to thereby accurately detect the temporal change of a phase difference signal. More specifically, an electromagnetic wave transmitting and receiving unit irradiates a region to be measured with an electromagnetic wave that is given strong directivity while scanning the region to be measured, receives a reflected wave, and acquires a phase difference signal corresponding to each position of the region to be measured, and the temporal change of the phase difference signal is detected.

### RELATED APPLICATIONS

This patent application is a continuation of International Application No. PCT/JP2012/054537, filed on Feb. 24, 2012, entitled, “LIVING ORGANISM INFORMATION DETECTION SYSTEM”, which claims priority to Japanese Patent Application No. 2011-039954, filed on Feb. 25, 2011, the contents and teachings of each of which are hereby incorporated by reference in their entirety.

### TECHNICAL FIELD

The present invention relates to a living organism information detection system that radiates an electromagnetic wave to a region of an object to be measured, receives a reflected wave, obtains a phase difference signal between an radiated wave and the reflected wave, and detects a condition of a living organism in the region of the object to be measured, based on the phase difference signal.

### BACKGROUND ART

There has conventionally been well-known a method in which an electromagnetic wave is radiated to an object to be measured, Doppler shift of the electromagnetic wave reflected by the object to be measured is utilized to determine an oscillation condition or displacement of the object to be measured. Especially, an electromagnetic wave having a microwave/milliwave bandwidth has also a nature of transmission in a medium such as dielectric, etc. It has recently been proposed to make an attempt to detect beat of a heart or a dynamic state of aspiration, which may be represented as oscillation in a human body, by radiating the electromagnetic wave to the human, utilizing such a nature.

Use of such an electromagnetic wave permits measurement, which may be performed in a non-contact state with a human body with his/her clothes on, neither performing a measurement in which detection electrodes come into a direct contact with the human in a restraint state, nor giving a new stress such as being more aware of measurement, thus providing an effect of minimizing the burden to be borne by a person to be measured to the lowest possible level.

An example of a measurement system utilizing such an electromagnetic wave is disclosed in JP 2002-58659 A or JP 2009-55997 A.

### CITATION LIST

### Patent Literature

PATENT LITERATURE 1: JP 2002-58659 A
PATENT LITERATURE 2: JP 2009-55997 A

### SUMMARY OF INVENTION

### Technical Problem

The conventional measurement system utilizing an electromagnetic wave, which is disclosed in each of the patent documents as indicated above, has a configuration in which the electromagnetic wave having a microwave bandwidth is used to detect a very small motion of a person to be measured, thus obtaining information such as heart-beat. More specifically, a micro-motion of a body surface of the person to be measured, which is caused based on oscillation such as the heart-beat of the person to be measured, is to be detected by detecting a temporal variation in phase of a reflected wave relative to a radiated wave. In this case, an accurate detection of such a phase variation provides the key to obtainment of accurate information such as the heart-beat.

As for the detection of the phase variation, it is preferable to radiate an electromagnetic wave to a place in which a signal including such a phase variation may be detected at a high level, for example, in case of information of the heat-beat to be obtained, to a chest, and cause the electromagnetic wave to be reflected by it, and then receive the reflected wave. In case where an antenna with a high directivity is used as an antenna for radiating and receiving the electromagnetic wave, difference in physical size of a respective person to be measured, or motion of the person to be measured may cause the radiation position of the electromagnetic wave to be deviated from the place in which the signal may be detected at a high level, with the result that a reflected wave based on which the phase variation may be detected, may not be obtained.

Therefore, there has conventionally taken measures that an antenna with a low directivity, for example, an omnidirectional antenna is normally used to transmit and receive an electromagnetic wave to expand a range of measurement, so that the electromagnetic wave as radiated surely reaches a part of a person to be measured, in which a phase variation based on motion of an object to be measured may surely be obtained from a reflected wave, and then the reflected wave may be received.

In this case, the expanded range of measurement however leads to a decreased level of the reflected wave as reflected by a certain part, with the result that the level of the signal including a phase variation, which may be obtained from the reflected wave, becomes also low.

In addition, when an object to be measured is normally not kept in a complete static condition in the same manner as a living organism, and always involves any motion, any other motion than the part to be measured of the object to e measured becomes noise, and the oscillation component in the reflected wave remarkably varies, and the remarkable variation of this oscillation component may have an influence on the detection of the phase variation. Therefore, the decreased level of the signal including the phase variation may lead to such a strong influence, thus causing a problem that the phase variation as detected may not effectively be used as one indicative of the motion such as the heart-beat or aspiration of the object to be measured.

Further, a wide range of radiation of the electromagnetic wave by an antenna may lead to a case where a plurality of living organisms unintentionally exists in the range of radiation. When the plurality of living organisms exists in the range of radiation in this manner, the reflected waves from every living organisms reach the antenna. Therefore, it is not possible to detect the phase variation based on the motion of the living organism of the object to be measured, distinctively from the other, thus causing a problem of difficulty even in recognizing whether a single living organism exists alone in the range of radiation or a plurality of living organisms exists in it.

An object of the present invention, which was made in order to solve the above-described problems, is to provide a living organism information detection system in which an electromagnetic wave is radiated, while scanning it, so as to ensure a proper performance of transmittance and receipt of the electromagnetic wave relative to a living organism within a region of an object to be measured, and a temporal variation of a phase difference signal may surely be detected so as to permit a proper obtainment of a desired information such as an existence of the living organism or a condition of the living organism.

### Solution to Problem

The living organism information detection system according to the present invention comprises: an electromagnetic wave transmitting and receiving unit that radiates an electromagnetic wave having a predetermined continuous frequency with a predetermined narrow directivity to a region of an object to be measured, and receives a reflected wave, and outputs a phase difference signal between an radiated wave and the reflected wave at respective positions of a whole of the region by an execution with a scanning of radiation and receipt; and a signal analyzing unit that analyzes the phase difference signal in correspondence with respective radiated positions as scanned in the region of the object to be measured, and, in case where there is a temporal variation in the phase difference signal, detects, one or a plurality of predetermined positions corresponding to a range of a time direction of the signal in which the variation has occurred, as an existing position of a living organism, and detects the temporal variation in the phase difference signal corresponding to the existing position, as a living organism information indicative of a condition of the living organism existing in the existing position.

According to the present invention, the electromagnetic wave transmitting and receiving unit radiates the electromagnetic wave with a high directivity, while scanning the region of the object to be measured, and receives the reflected wave, so as to obtain the phase difference signal corresponding to the respective positions of the region of the object to be measured, and the signal analyzing unit detects the temporal variation of the phase difference signal as the living organism information. This makes it possible to ensure radiation of the electromagnetic wave to the living organism during a scanning operation to receive the reflected wave from the living organism, and enhance a signal intensity of the phase difference signal including a temporal variation indicative of the motion of the living organism to ensure a detection of the temporal variation, thus permitting a precise recognition of the existence of a single or a plurality of the living organisms within the region of the object to be measured, the existing position or the range of existence of them. The detection of the existence of a single or a plurality of living organisms within the region of the object to be measured permits to obtain the size of the living organism in a non-contact state or detect the existence or the existing position of the living organism in a state in which the living organism may not directly be visually recognized in a predetermined region, thus being utilized for security measures or confirmation of survivals in a scene of a disaster.

The living organism information detection system according to the present invention may comprise, where appropriate: a living organism information processing unit that obtains a peak component of a signal, which is generated substantially periodically in response to a roughly steady micro-motion representing vital signs of a human as the living organism, from the temporal variation in the phase difference signal as the living organism information as detected by the signal analyzing unit, to determine an appearance interval information of the vital signs, wherein: the electromagnetic wave transmitting and receiving unit further radiates the electromagnetic wave to the one or the plurality of existing positions corresponding to the living organism information as detected by the signal analyzing unit, while continuing the scanning, and maintains a state in which the reflected wave is to be received, for a predetermined period of time until the phase difference signal having a signal length necessary for determination of the appearance interval information may be obtained by the living organism information processing unit, and outputs newly a phase difference signal, the signal analyzing unit detects the temporal variation in the phase difference signal as the living organism information for the respective existing positions, from the phase difference signal as newly outputted, and the living organism information processing unit determines the appearance interval information of the vital signs for the respective living organism, from the temporal variation in the phase difference signal for the respective existing positions.

According to the present invention, the electromagnetic wave transmitting and receiving unit newly conducts the radiating and receiving operation of the electromagnetic wave for a predetermined period of time, for each of the existing positions of the living organism, as obtained once, the signal analyzing unit detects the temporal variation of the scanning signal as outputted, and the living organism information processing unit determines an appearance interval information of vital signs for each of the living organisms, for example, information such as a heart-beat interval or a pulse interval. This makes it possible to provide a more detailed recognition of a condition of the living organism existing in the region of the objet to be measured. In addition, the appearance intervals of the vital signs such as the heart-beat interval or the pulse interval of each of the living organisms differ bit by bit, with result that it is possible to distinguish the living organisms from each other and precisely recognize the number of living organisms existing in the region of the objet to be measured.

The living organism information detection system according to the present invention may comprise, where appropriate: a recording unit that records as a database for the respective living organism, the appearance interval information of the vital signs as obtained by the living organism information processing unit, together with the existing position; and a cross-checking unit that makes a cross-checking between the living organism information as recorded by the recording unit and the appearance interval information of the vital signs for the respective living organism as newly obtained by the living organism information processing unit to identify the living organism.

According to the present invention, the recording unit records the appearance interval information of the vital signs for each of the living organisms, for example, information such as the heart-beat interval or the pulse interval, together with the existing position of the living organism, and the cross-checking unit makes the cross-checking between the information as recorded and the information as newly obtained by the living organism information processing unit to identify the living organism, thus permitting to determine that which living organism exists in which position in the region of the object to be measured. This makes it possible to distinguish and identify, even when living organisms come into the region of the object to be measured and go out of it, the living organism every time they come and go, and to track and monitor the state of the living organism even when the living organism may not directly be visually recognized.

The living organism information detection system according to the present invention may have, where appropriate, a feature that the scanning in the electromagnetic wave transmitting and receiving unit is achieved through radiation by a phase control of an array antenna or deflection of a receiving direction.

According to the present invention, the scanning in the electromagnetic wave transmitting and receiving unit is achieved by the phase control of an array antenna, and the process in which the electromagnetic wave is radiated to the living organism with a high directivity as given and the reflected wave is received, without moving any part of the electromagnetic wave transmitting and receiving unit, is performed in the whole of the region of the object to be measured, with the result that there is no need to provide a movable part and the electromagnetic wave transmitting and receiving unit may be achieved with a simple structure. In addition, a space for movement for the scanning is made redundant through no movement of the antenna, to make the occupied space small, and the antenna may be provided in a barely noticeable position, thus making, in case where the living organism is a human, him/her barely aware of the existence of the antenna.

### BRIEF DESCRIPTION OF DRAWINGS

FIG. 1 is a block diagram of a living organism information detection system according to an embodiment of the present invention;

FIG. 2 is a descriptive view of a region of an object to be measured of the living organism information detection system according to the embodiment of the present invention;

FIG. 3 is a descriptive view of another example of the living organism information detection system according to the embodiment of the present invention;

FIG. 4 is a graph of a temporal variation in a phase difference signal as obtained in a radiation state in Example 1 of the living organism information detection system according to the present invention;

FIG. 5 is a graph of a frequency power spectrum of the phase difference signal as obtained in the radiation state in Example 1 of the living organism information detection system according to the present invention;

FIG. 6 is a graph of a temporal variation in a phase difference signal as obtained in a radiation state in Example 2 of the living organism information detection system according to the present invention;

FIG. 7 is a graph of a frequency power spectrum of the phase difference signal as obtained in the radiation state in Example 2 of the living organism information detection system according to the present invention;

FIG. 8 is a graph of a temporal variation in a phase difference signal as obtained in a radiation state in Example 3 of the living organism information detection system according to the present invention;

FIG. 9 is a graph of a frequency power spectrum of the phase difference signal as obtained in the radiation state in Example 3 of the living organism information detection system according to the present invention;

FIG. 10 is a graph of a temporal variation in a phase difference signal as obtained in a radiation state in Example 4 of the living organism information detection system according to the present invention;

FIG. 11 is a graph of a frequency power spectrum of the phase difference signal as obtained in the radiation state in Example 4 of the living organism information detection system according to the present invention;

FIG. 12 is a graph of a temporal variation in a phase difference signal as obtained in a radiation state in Example 5 of the living organism information detection system according to the present invention;

FIG. 13 is a graph of a frequency power spectrum of the phase difference signal as obtained in the radiation state in Example 5 of the living organism information detection system according to the present invention;

FIG. 14 is a graph of a temporal variation of a signal as obtained by an electrocardiograph as Comparative Example 1 in comparison with the living organism information detection system according to the present invention;

FIG. 15 is a graph of a frequency power spectrum of the signal as obtained by an electrocardiograph as Comparative Example 1 in comparison with the living organism information detection system according to the present invention;

FIG. 16 is a graph of a temporal variation of a signal as obtained by an electrocardiograph as Comparative Example 2 in comparison with the living organism information detection system according to the present invention;

FIG. 17 is a graph of a frequency power spectrum of the signal as obtained by an electrocardiograph as Comparative Example 2 in comparison with the living organism information detection system according to the present invention;

FIG. 18 is a graph of a temporal variation of a signal as obtained by an electrocardiograph as Comparative Example 3 in comparison with the living organism information detection system according to the present invention;

FIG. 19 is a graph of a frequency power spectrum of the signal as obtained by an electrocardiograph as Comparative Example 3 in comparison with the living organism information detection system according to the present invention;

FIG. 20 is a graph of a temporal variation of a signal as obtained by an electrocardiograph as Comparative Example 4 in comparison with the living organism information detection system according to the present invention;

FIG. 21 is a graph of a frequency power spectrum of the signal as obtained by an electrocardiograph as Comparative Example 4 in comparison with the living organism information detection system according to the present invention;

FIG. 22 is a graph of a temporal variation of a signal as obtained by an electrocardiograph as Comparative Example 5 in comparison with the living organism information detection system according to the present invention; and

FIG. 23 is a graph of a frequency power spectrum of the signal as obtained by an electrocardiograph as Comparative Example 5 in comparison with the living organism information detection system according to the present invention.

### DESCRIPTION OF EMBODIMENTS

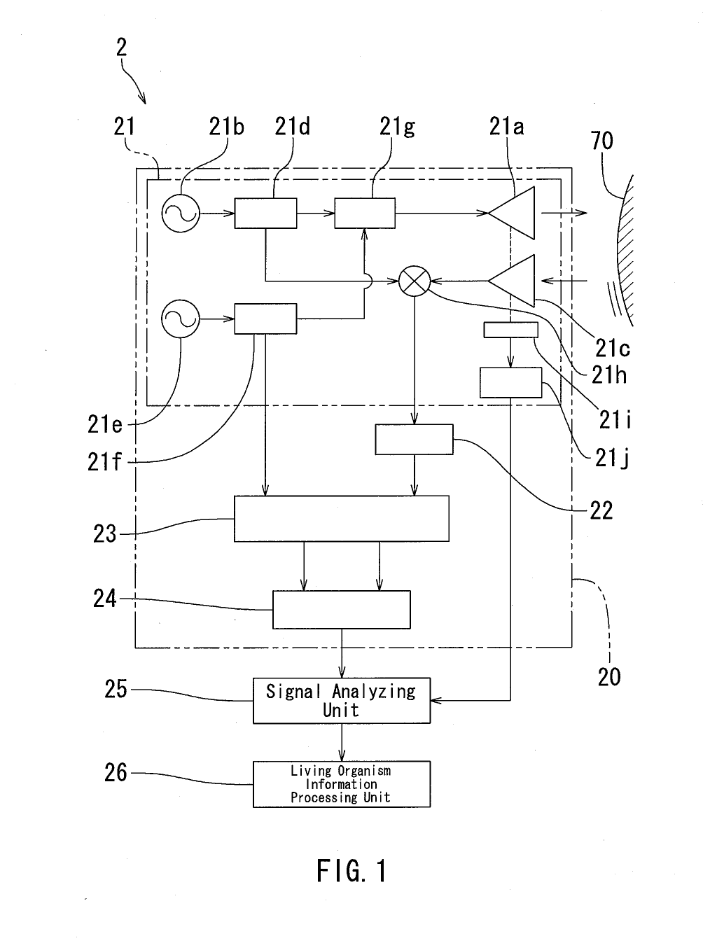

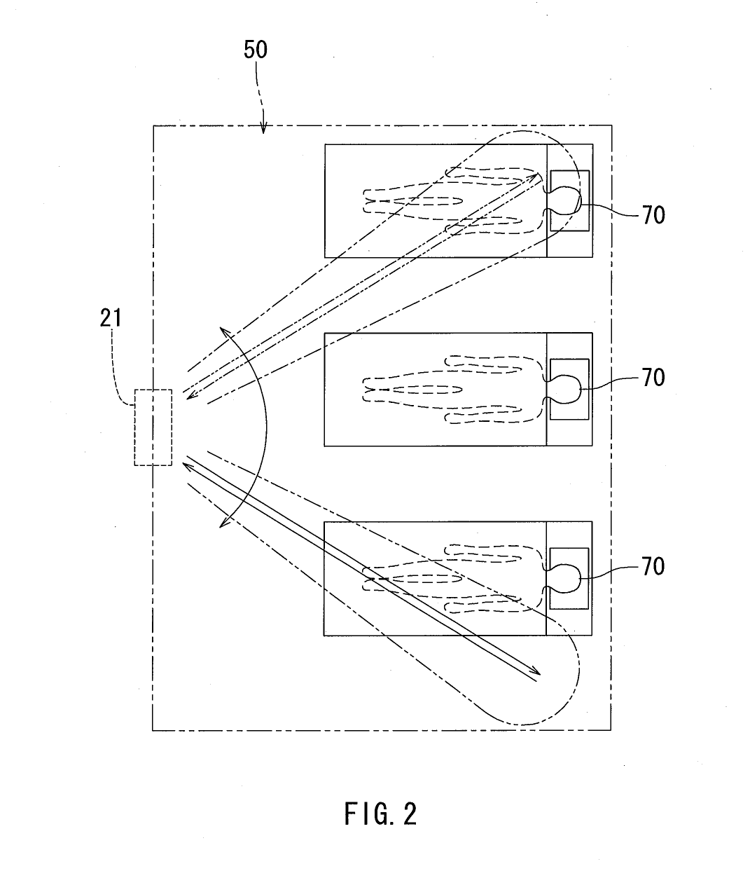

Now, a living organism information detection system according to an embodiment of the present invention will be described below with reference to FIG. 1 and FIG. 2 as indicated above.

The living organism information detection system 2 according to the embodiment of the present invention in the above respective views is provided with an electromagnetic wave transmitting and receiving unit 20, a signal analyzing unit 25 and a living organism information processing unit 26, and has a configuration that the electromagnetic wave transmitting and receiving unit 20 uses a radiating antenna 21a and a receiving antenna 21c, and generates a radiation wave or a reference wave in heterodyne using a microwave oscillator 21b, as well as a high-frequency oscillator 21e and an up converter 21g, and radiates an electromagnetic wave having a microwave bandwidth to a region 50 of an object to be measured such as a room having a space in which a plurality of humans 70 as a living organism may exist, while scanning it, and receives a reflected wave.

The electromagnetic wave transmitting and receiving unit 20 is provided with a transmitting and receiving section 21 that radiates a microwave to the region of the object to be measured and receives a reflected wave, and outputs a reflected wave signal and a reference signal for quadrature detection, an adjusting unit 22 that adjusts a signal output level for the reflected wave signal as outputted from the transmitting and receiving section 21, a quadrature detecting unit 23 that performs a quadrature detection processing with the use of the reflected wave signal as adjusted by the adjusting unit 22 and the reference signal as indicated above, and obtains a signal of the in-phase component with the reflected wave and a signal of the orthogonal component to it, and a calculating unit 24 that calculates a phase difference signal between the radiated wave and the reflected wave, from the in-phase component signal and the orthogonal component signal as outputted from the quadrature detecting unit 23.

The above-mentioned transmitting and receiving section 21 is provided with a radiating antenna 21a that radiates the microwave to the region 50 of the object to be measured, a microwave oscillator 23b that generates the microwave for generating the radiating wave from the above-mentioned radiating antenna 21a, a receiving antenna 21c that receives the reflected wave, a directional coupler 21d that separates the microwave generated by the microwave oscillator 21b into a component for generation of the radiating wave and a component for generation of the reflected wave signal, a high-frequency oscillator 21e that generates a high-frequency (VHF or UHF bandwidth) of an intermediate frequency, which is to be a reference eave signal, a distributor (a power splitter) 21f that distributes the high-frequency as generated by the high-frequency oscillator 21e into a component for generation of the radiating wave and a component for the reference wave signal, an up convertor 21g that generates the radiated wave, which is to be a microwave as shifted from the original microwave by an amount of the intermediate frequency, a mixer section 21h that obtains the reflected wave signal from the reflected wave as received by the receiving antenna 21c and the microwave passing through the directional coupler 21d, an antenna driving mechanism 21i that changes the directions of the respective antennas 21a, 21c relative to the region of the object to be measured, to conduct the radiation of the microwave in the scanning state, and an antenna controlling section 21j that controls this antenna driving mechanism 21i.

Of the respective signal outputted from the transmitting and receiving section 21, the reflected wave signal of the intermediate frequency as obtained by the mixer section 21h is outputted to the adjusting unit 22. The reference wave signal of the intermediate frequency, which has been generated by the high-frequency oscillator 21e and passed through the distributor 21f, is outputted to the quadrature detecting unit 23.

In the transmitting and receiving section 21, there is adopted the heterodyne that uses only a single microwave oscillator 21b and uses the high-frequency oscillator 21e and the up converter 21g together to generate the radiated wave and the reference wave. In the heterodyne in which two microwave oscillators are provided, the use of the two microwave oscillators provides stability in which the fluctuations of the both oscillators are superimposed. To the contrary, when the above-mentioned up convertor 21g is used, the stability is determined by the fluctuation of only the high-frequency oscillator 21e, thus making it possible to make the fluctuation component small and improve an accuracy of the phase measurement.

The above-mentioned radiating antenna 21a and the receiving antenna 21c, which are a waveguide antenna, which is suitable for the microwave bandwidth, and the direction to the region 50 of the object to be measured may be changed by the antenna driving mechanism 21i. There may be adopted a configuration that an array antenna is used as these antennas and an amount of deviation of the radiated wave from the respective antennas serving as the array antenna is adjusted and controlled, thus permitting a scanning through the whole of the antennas.

As for a guide for characteristic properties of the directivity of these antennas, it is preferable to keep the beam width of the radiated wave within about 100 cm as a size, which may be individually caught by each person within the region, in each part of the region of the object to be measured, which is placed apart from the antenna by a predetermined distance.

In this case, the antenna is designed as a narrow directivity having the above-mentioned beam width and an absolute gain of the main lobe is 25 dBi or more. This makes it possible to provide an enough power density for measurement, which exceeds the minimum power density by which the reflected wave signal receivable in the system, while controlling the radiation output power, which may ensure safety relative to the health damage through the whole system

The above-mentioned antenna driving mechanism 21i mechanically moves a base portion on which the radiating antenna 21a and the receiving antenna 21c are mounted, to change the direction of the respective antenna relative to the region 50 of the object to be measured, thus providing a configuration that the scanning operation can be performed by changing the directions of the radiation of the electromagnetic wave and the receipt of the reflected wave through the respective antennas.

The above-mentioned antenna controlling section 21j controls the antenna driving mechanism 21i so as to orient the radiating antenna 21a and the receiving antenna 21c in the proper directions. More specifically, the measurement of the whole of the region 50 of the object to be measured can be performed by making control so as to repeat, in a state in which the radiating antenna 21a and the receiving antenna 21c are oriented in the predetermined directions, the steps of causing the radiating antenna 21a to radiate the electromagnetic wave in its direction and of causing the receiving antenna 21c to receive the reflected wave until there has lapsed a predetermined period of time during which the reflected wave from a human 70 in the region 50 of the object to be measured may reach, while changing slightly the directions of the respective antennas by the antenna driving mechanism 21i, and performing the scanning operation.

The information on the temporal variation of the radiation direction, which is outputted from the antenna controlling section 21j, is used to obtain a relationship between the temporal variation of the phase difference signal and the radiation position within the region 50 of the object to be measured, in the signal analyzing unit 25.

The above-mentioned adjusting unit 22 adjusts the signal output level of the reflected wave signal outputted from the transmitting and receiving section 21 to obtain the reflected wave signal within a predetermined output range. Giving a more detailed description, the adjusting unit 22 is provided with a gain variable amplifier and a detection controller so as to provide a configuration that the output from the gain variable amplifier is detected and monitored by the detection controller, and a gain of the gain variable amplifier is controlled so as to become a constant output as previously set, thus performing a so-called AGC (Automatic Gain Control).

The reflected wave signal, which has been adjusted to the predetermined level by the adjusting unit 22, is inputted, together with the reference eave signal of the intermediate frequency as distributed by the distributor 21f, to the quadrature detecting unit 23, thus being subjected to the quadrature detection processing. There may be adopted a configuration that, in addition to the adjustment based on the output from the gain variable amplifier, the adjusting unit 22 sends a control instruction from the calculating unit 24 to the adjusting unit 22 so that the respective amplitude components of the in-phase component signal and the orthogonal component signal to be inputted to the calculating unit 24 are kept in the appropriate range, thus making adjustment of the signal output level.

Adjusting constantly the output level of the reflected wave signal by the adjusting unit 22 may prevent a large variation in the amplitude component between the in-phase component signal and the orthogonal component signal, as obtained by the subsequent quadrature detecting unit 23, the calculation of the phase variation by the calculating unit is not subject to influence on the variation of the amplitude component, thus permitting to obtain an appropriate value.

The above-mentioned quadrature detecting unit 23 performs the quadrature detection processing, utilizing the reflected wave signal as adjusted by the adjusting unit 22 and the reference wave signal of the intermediate frequency as outputted from the transmitting and receiving section 21, to obtain the signal, which is of the in-phase component with the reflected wave signal, and the signal, which is of the orthogonal component to it, which may be obtained by a commonly used microwave receiving circuit.

The quadrature detecting unit 23 demodulates, as the quadrature detecting processing, the reference wave signal (A cos ωt) and the reflected wave signal (B cos(ωt+Δφ) in combination, to obtain the in-phase component signal (Eᵣ cos Δφ) and the orthogonal component (Eᵣ sin Δφ) of the phase variation. Obtainment of these signals enables the calculating unit 24 to perform a simple calculation processing to make a separation between the amplitude component Eᵣ and the phase difference component Δφ to obtain the phase difference signal. The amplitude component Eᵣ is a product of the amplitude A of the reference wave signal and the amplitude B of the reflected wave signal.

The above-mentioned calculating unit 24 calculates the phase difference signal between the radiated wave and the reflected wave (the component, which is directly in proportion to the phase variation) from the in-phase component signal and the orthogonal component signal, as described above, which have been outputted from the quadrature detecting unit 23. Giving a more detailed description, the component, which is directly in proportion to Δφ, may be calculated based on the following relationship:

Δφ=tan⁻¹(Eᵣ sin Δφ/Eᵣ cos Δφ),

, using the in-phase component signal (Eᵣ cos Δφ) and the orthogonal component (Eᵣ sin Δφ) of the phase variation Δφ as obtained by the quadrature detecting unit 23, and make a separation between the amplitude (the component Eᵣ) of the signal and the phase (the component φ), thus obtaining the phase difference signal. The phase variation Δφ corresponds to an amount of movement of a reflection surface in the region of the object to be measured, and consequently, the phase difference signal may vary along with a displacement of the reflection surface.

In the human as the living organism, which exists in the region 50 of the object to be measured to which the microwave is to be radiated, it is not kept in a complete static condition due to the living organism, even when he/she does not move from there, and micro-motion of muscle or the like may occur in some parts of the body. Variation in a position on the body surface, caused by such a motion, is reflected in the phase variation Δφ as described above, with the result that the phase difference signal as obtained may include the temporal variation. Therefore, it is possible to recognize, from the temporal variation of the phase difference signal, the existence of a human as the living organism, or the existing position in the region of the object to be measured.

The temporal variation of the phase difference signal includes a peak component, which corresponds to a roughly steady micro-motion representing vital signs of a human. The peak component appears periodically in response to the micro-motion. An appearance interval of the vital signs, e.g., an interval of a heart-beat interval or a pulse interval, or a blink, etc., may be determined from the interval of substantially the periodical peak component of the temporal variation of the phase difference signal.

The above-mentioned signal analyzing unit 25 analyzes the phase difference signal in association with the respective radiation positions as scanned in the region 50 of the object to be measured, and detects, in case where the temporal variation of the phase difference signal occurs, a single of a plurality of predetermined positions in the region 50 of the object to be measured, which corresponds to a range of direction of time of the signal, in which such a variation occurs, as an actually existing position of a human, and detects the temporal variation of the phase difference signal corresponding to the existing position, as a living organism information indicative of a condition of the living organism existing in the existing position as mentioned above.

The detection of the temporal variation of the phase difference signal as the living organism information by the above-mentioned signal analyzing unit 25 permits to recognize the existence of a single or a plurality of the humans within the region of the object to be measured, or the existing position of them.

The above-mentioned living organism information processing unit 26 further obtains, from the temporal variation of the phase difference signal as the living organism information as detected by the signal analyzing unit 25, substantially the periodical peak component of the signal, which is generated in response to the roughly steady micro-motion representing the vital signs of a human, for example, a heart-beat interval or a pulse interval, or a blink, etc., and determines the appearance interval information of the vital signs corresponding to the interval of the occurrence of the peak component, for example, information on the interval of a heart-beat interval or a pulse interval, or a blink, etc.

In addition to the living organism information processing unit 26, the calculating unit 24 and the antenna controlling section 21j of the above-mentioned electromagnetic wave transmitting and receiving unit 20, as well as the signal analyzing unit 25 constitute, as a hardware structure, a computer provided with a CPU, a memory, input and output interfaces, etc, and cause the computer as the calculating unit 24, the antenna controlling section 21j, the signal analyzing unit 25 and the living organism information processing unit 26 as described above, by a program stored in the memory, etc. Incidentally the calculating unit 24, the antenna controlling section 21j, the signal analyzing unit 25 and the living organism information processing unit 26 may constitute independently or in combination a plurality of computers. Such a computer may be a microcomputer integrally provided with a CPU, a memory, a ROM, etc.

Now, description will be given below of a state of use of the living organism information detection system according to the embodiment of the present invention. There is an assumption that a single or a plurality of humans 70 as the living organism exist in a room space serving as the region 50 of the object to be measured, and any other substance than the living organism is kept in a static environment in the region 50 of the object to be measured, and the human 70 is not put under constraint and kept in a state (a non-static state) in which a motion is permitted. In addition, the living organism information processing unit 26 determines information on the heart-beat interval as the vital signs.

The transmitting and receiving section 21 of the electromagnetic wave transmitting and receiving unit 20 radiates the continuous microwave from the antenna 21a to the region 50 of the object to be measured for a predetermined period of time and receives the reflected wave by the receiving antenna 21c, while performing the scanning operation of changing the directions of the radiating antenna 21a and the receiving antenna 21c by the antenna driving mechanism 21i, and outputs the phase difference signal between the radiated wave and the reflected wave at the respective positions in the whole of the region 50 of the object to be measured. This phase difference signal and the information on the temporal variation of the radiation direction, as simultaneously outputted from the antenna controlling section 21j are inputted to the signal analyzing unit 25.

The signal analyzing unit 25 analyzes the phase difference signal as obtained in association with the respective radiation positions in the region 50 of the object to be measured, and detects a position in the region 50 of the object to be measured, which corresponds to a range, in which the temporal variation occurs along with a motion of the human 70, as an existing position of the human 70, and detects the temporal variation of the phase difference, as a living organism information indicative of a condition of the living organism existing in the existing position as mentioned above. The detection of the temporal variation of the phase difference signal as the living organism information permits to recognize the existence of the human within the region of the object to be measured, or the existing position of it.

After recognition of the existing position of the human, the electromagnetic wave transmitting and receiving unit 20 further radiates the electromagnetic wave to the single or the plurality of existing positions in the region 50 of the object to be measured, while performing the scanning operation, and continuously keeps a state to receive the reflected wave for a predetermined period of time at the respective existing position, more specifically, for a period of time necessary for obtainment of the phase difference signal having the signal length required to obtain information on the heart-beat interval from the interval of substantially the periodical peak component as generated in response to the heart-beat in the living organism information processing unit 26, and then newly outputs the phase difference signal.

The signal analyzing unit 25 detects the temporal variation of the phase difference signal as the living organism information at the respective existing position, from the phase difference signal as newly outputted. In addition, the living organism information processing unit 26 obtains, from the temporal variation of the phase difference signal at the respective existing position, substantially the periodical peak component of the signal, which is generated in response to the heart-beat of the human, and determines the appearance interval information of the vital signs corresponding to the interval of the occurrence of the peak component. It is possible to recognize a condition as the heart-beat interval for a single of a plurality of humans existing in the region of the object to be measured, in this manner.

The obtainment of the heart-beat interval by the living organism information processing unit 26 permits to further determine a heart-beat interval variation (HRV). When a stress analyzing device is independently used to make sequentially a frequency analysis of the heart-beat interval variation, there may be provided a state in which the temporal variation of respective spectrum peaks in a bandwidth (LF component) of about 0.03 to 0.15 Hz and a bandwidth (HF component) of 0.15 to about 0.45 Hz may be extracted for a relatively short period of time. Utilizing this also permits to perform a stress assessment for a single or a plurality of humans in the region of the object to be measured. In the stress assessment, a period of time in which the peak of LF appears stronger than HF, may be deemed as a state in which a stress is given to the human, and a period of time in which the peak of HF appears stronger than LF, may be deemed as a relaxed state of the human, thus making it possible to make an assessment for a short period of time.

In the stress assessment based on the heart-beat interval variation, it is possible to make a measurement in a non-contact state with a body of the human, to capture surely the heart-beat interval, without being aware of measurement and giving to the human factors to deteriorate the measurement accuracy, such as tension or the like, thus performing a proper stress assessment and improving the assessment accuracy.

As for an example of the application of the living organism information detection system according to the embodiment of the present invention, it is possible to recognize the existence of the human in the region of the object to be measured, and its existing position. If a place where any person should not exist is set as the region of the object to be measured, there may be provided a system of detecting an intruder. If a scene of an accident, a disaster or the like is set as the region of the object to be measured, the system may also be applied for confirmation of the existence (surviving) or the existing position of a human who has been buried alive due to such an accident or disaster and kept in a state that the human may not directly be visually recognized. Concerning a place where a security issue is emphasized and not only general public, but also suspicious individuals or criminals such as terrorists may be anticipated to enter, if such a place is set as the region of the object to be measured, it is possible to recognize the existence and the existing position of the humans in the region, and measure a variation of the condition of the vital signs such as the heart-beat for the respective human and further make a stress assessment based on the heart-beat interval. Consequently, there may be provided a system in which subtle symptoms of the stress may be distinguished through such a stress assessment, so as to detect a suspicious person and promote appropriate responses of security workers, or the like.

If the transmitting and receiving section (antennas) 21 of the electromagnetic wave transmitting and receiving unit 20 is placed, as a monitor for condition of health, in a place where the electromagnetic wave may be radiated to the whole room, for example on a ceiling or wall, in a room space for daily living, a hospital room, or the like, which may be the region of the object to be measured in which a single or a plurality of humans may be expected to exist, it is possible to provide a system which permits to recognize the existence and the existing position of the human in the region of the object to be measured and measure a so-called variation of the condition of the vital signs such as the heart-beat, aspiration, or the like of the human during a physical activity or sleep for each human, thus making an assessment of the health condition (see FIG. 2). Alternatively, if the respective antennas of the electromagnetic wave transmitting and receiving unit are embedded, as a monitor to recognize the number of fellow passengers in an automobile, in predetermined places where the electromagnetic wave may be radiated to the whole room of the automobile, it is possible to detect the temporal variation of the phase difference signal, without especially putting the driver or the fellow passengers under constraint, to recognize the existence and the existing position of the human in the automobile as the region of the object to be measured, thus permitting to obtain the number of the humans in the automobile. The above-mentioned number of the humans may effectively be utilized as information for a driving control of a power unit or an output control for an air conditioner. If the stress assessment is additionally made, determination of the driver based on the existing position of the human existing in the room of the automobile and performance of the stress assessment based on the heart-beat or the like of the driver during driving, permit to provide a system of permitting to provide information on a guide for a break or rest of the driver, a warning of drowsy driving.

In addition to it, the present invention may be applied to a stress analysis system in which a space that is a working environment for a single or a plurality of workers is set as the region of the object to be measured, the respective antennas of the electromagnetic wave transmitting and receiving unit are placed in this space, the temporal variation of the phase difference signal is detected, without putting the worker under constraint, to recognize the existence and the existing position of the human in the region of the object to be measured, and the heart-beat interval variation is introduced for each human to make a stress assessment during working, a degree of fatigue, etc., is assessed from the stress condition during the working period of time, thus using information as a guide for suitability for the operation of the worker or a guide for work break.

In the living organism information detection system according to the embodiment of the present invention, the electromagnetic wave transmitting and receiving unit 20 performs the radiation of the electromagnetic wave and the receipt of the reflected wave for a sufficient period of time, the signal analyzing unit 25 detects the temporal variation of the phase difference signal as obtained, and the living organism information processing unit 26 obtains information on the heart-beat interval. This makes it possible to provide a more detailed recognition of a condition of the human existing in the region 50 of the objet to be measured, and make an assessment of it, and distinguish the human due to the heart-beat interval being slightly different from each other, and recognize correctly the number of the humans existing in the region 50 of the object to be measured.

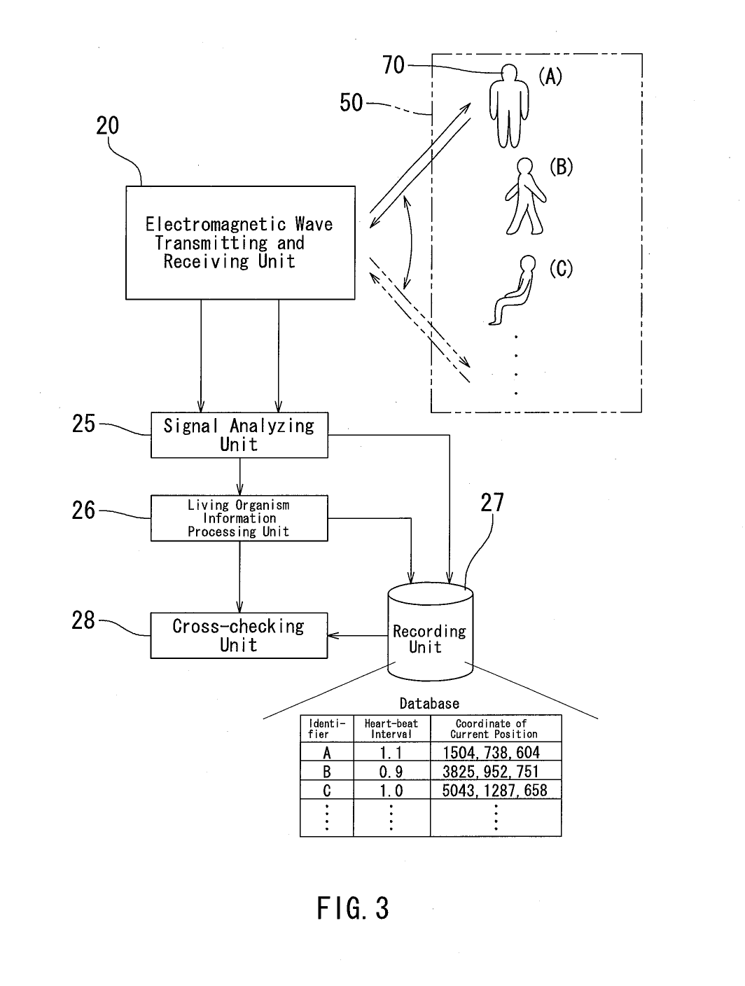

In the living organism information detection system according to the embodiment of the present invention as described above, there is applied a configuration in which the living organism information processing unit 26 determines the appearance interval information of the vital signs from the temporal variation of the phase difference signal, to recognize the condition of the human. However, there may be applied a configuration in which there are provided a recording unit 27 to record as a database for the respective living organism, the appearance interval information of the vital signs of the human 70 as the living organism as obtained by the living organism information processing unit 26, together with the existing position, and a cross-checking unit 28 to use the information of the living organism as recorded in the recording unit 27 to make a comparison and a cross-checking between the living organism information as recorded by the recording unit and the appearance interval information of the vital signs for the respective human as newly obtained by the living organism information processing unit 26 to identify the human, as shown in FIG. 3. In this case, the cross-checking unit 28 makes a cross-checking between the information as recorded in the recording unit 27 and the newly obtained information to identify the human, thus determining that which human exists in which position in the region of the object to be measured. This makes it possible to distinguish and identify, even when the humans come into the region of the object to be measured and go out of it, the human every time they come and go, and to track and monitor the state of the human even when the human may not directly be visually recognized.

### Example

With the use of the living organism information detection system of the present invention, a plurality of radiation states of the electromagnetic wave in conformity to the scanning operation, were determined, tests were made for a discrimination ability of the existence of the human as the living organism, from the reflected waves as obtained respectively, and in case where the human existed, and there was made a comparative assessment between the information on the heart-beat (the heart-beat frequency) as derived and the heart-beat frequency, as a comparative example, as determined from the results of the heart-beat measurement by an electrocardiograph.

More specifically, with the use of the living organism information detection system according to the present invention as described above, the radiation states of the electromagnetic wave in the region of the object to be measured were changed in conformity to the scanning operation, and in case where the human existed, the phase difference signal including the peak component corresponding to the heart-beat were obtained in the respective state, and the heart-beat frequency was obtained through the frequency analysis of the phase difference signal.

Concerning the radiation states, there were set five states, i.e., a state in which a brow of the human who was apart from the antenna by 1 m was located as a target of radiation of the electromagnetic wave in front of the front face of the antenna (Example 1), a state in which the antenna was shifted from the above-mentioned state so that only a part of the body of the human who was apart from the antenna by 1 m was included in the radiation range of the electromagnetic wave (Example 2), a state in which a brow of the human who was apart from the antenna by 2.5 m was located as a target of radiation of the electromagnetic wave in front of the front face of the antenna (Example 3), a state in which the antenna was shifted from the above-mentioned state so that only a part of the body of the human who was apart from the antenna by 2.5 m was included in the radiation range of the electromagnetic wave (Example 4), and a state in which no human existed in the radiation range of the electromagnetic wave from the antenna (Example 5). The phase difference signals were obtained in these states.

Both of the transmitting and receiving antennas of the electromagnetic wave transmitting and receiving unit for obtain the phase difference signal were a horn antenna. The electromagnetic wave radiated from the antenna was a microwave having the frequency of 10.525 GHz. The microwave was generated by the microwave oscillator and passed through the directional coupler and the up converter, and then radiated from the antenna.

The reflected wave, which was reflected from the human, etc., and received by the antenna, was passed through the mixer section and sent to the adjusting unit, to be subjected to a level adjustment, and then came into the quadrature detection unit. The quadrature detection unit obtained a signal component based on the phase variation, and the thus obtained signal component was processed by the calculating unit to output the phase difference signal. In case where the human existed on the front side of the antenna for radiating the microwave, the phase difference signal included the peak component of the motion (oscillation) on the reflection face on the surface of the body corresponding to the beat of the heart. Even when any human did not exist, the reflected wave from the wall, etc., was received to obtain the phase difference signal in the same manner as a case where the human existed.

The living organism information processing unit caused the phase difference signal as obtained by the electromagnetic wave transmitting and receiving unit to be subjected to a frequency analysis to obtain a power spectrum of the frequency (the heart-beat frequency) indicative of a occurrence frequency of the peak component as mentioned above, for the peak component of the signal as generated in response to the heart-beat of a body being tested, which was included in the phase difference signal. A value of the frequency of the maximum peak position (the spectrum peak) in the frequency range, which was actually applicable was recognized as the heart-beat frequency and determined as information on the heart-beat interval.

The measurement of the heart-beat was carried out by an electrocardiograph, at the same time as the obtainment of the signal by the radiation of the microwave. The measurement was conducted with electrodes of the electrocardiograph brought into a direct contact with a plurality of parts of a body of the human (a subject), in the same manner as a common measurement by the electrocardiograph. In each of a case of the radiation of the microwave and the receipt of the reflected wave, and a case of the measurement by the electrocardiograph, the measurement were carried out in a state in which the human took a seat and did not move in the region of the object to be measured, and the continuance was 30 seconds. However, only in case of Example 5 in which any human did not exist in the radiation region of the microwave, the continuance was 20 seconds.

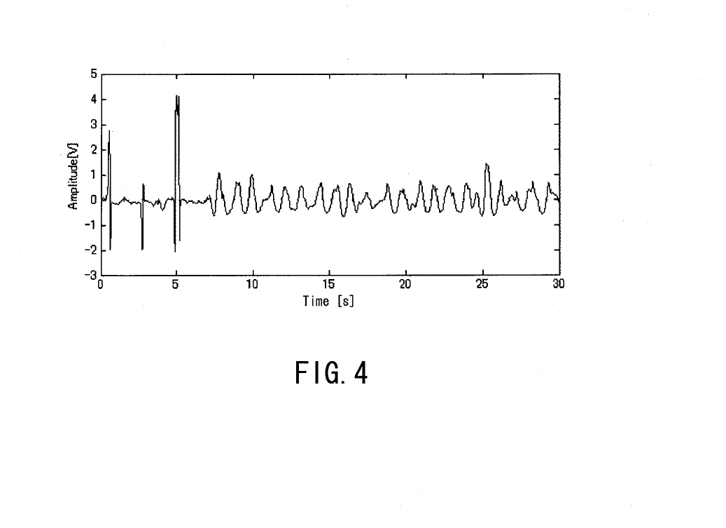

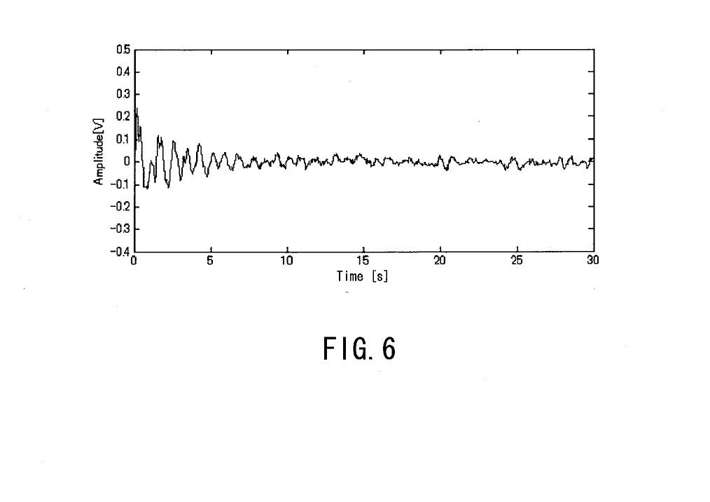

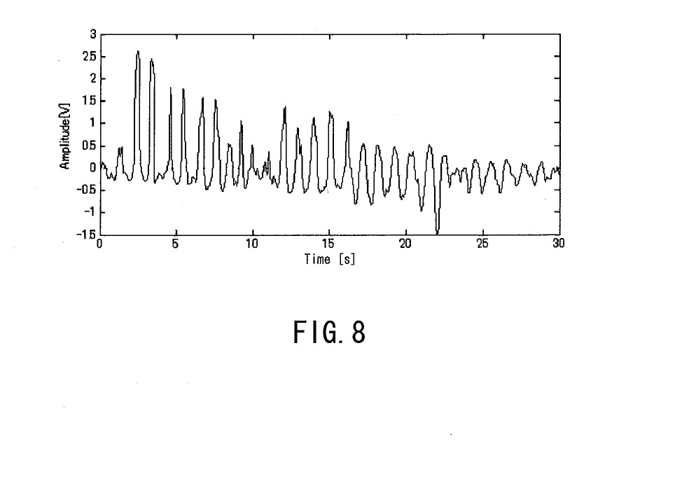

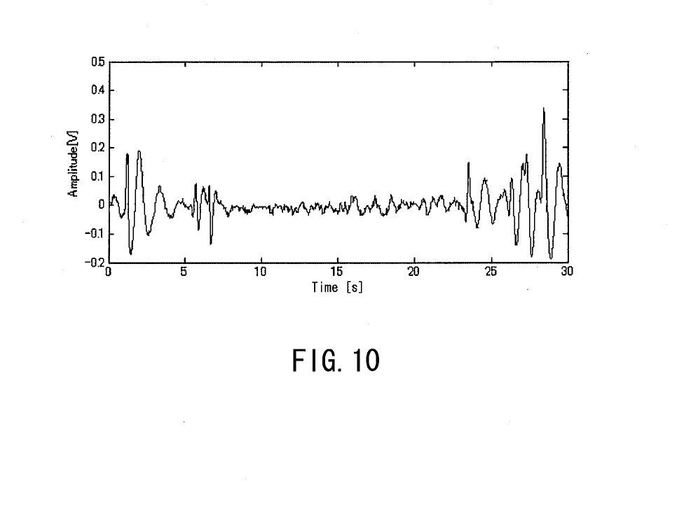

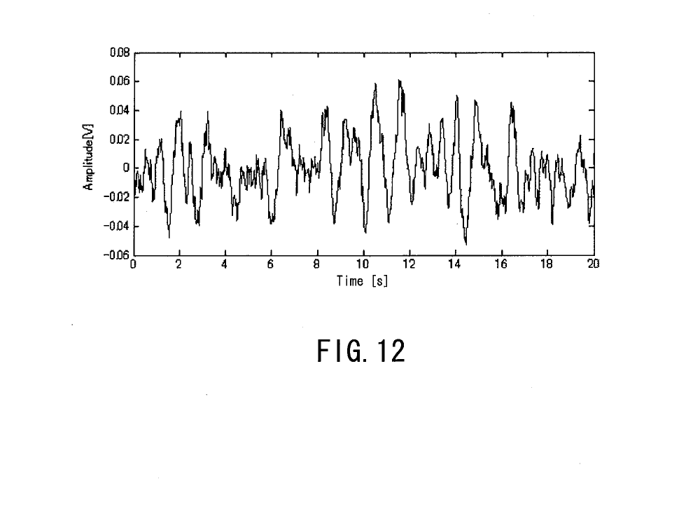

FIG. 4, FIG. 6, FIG. 8, FIG. 10 and FIG. 12 respectively show graphs in which the waveforms of the temporal variations of the phase difference signals, as obtained by the electromagnetic wave transmitting and receiving unit, including the peak components corresponding to the heart-beats in the case of the existence of the human in the respective states of Examples 1 to 5 as mentioned above, are plotted, wherein the abscissa of the graphs denotes a lapse time [s], and the ordinate, amplitude [V].

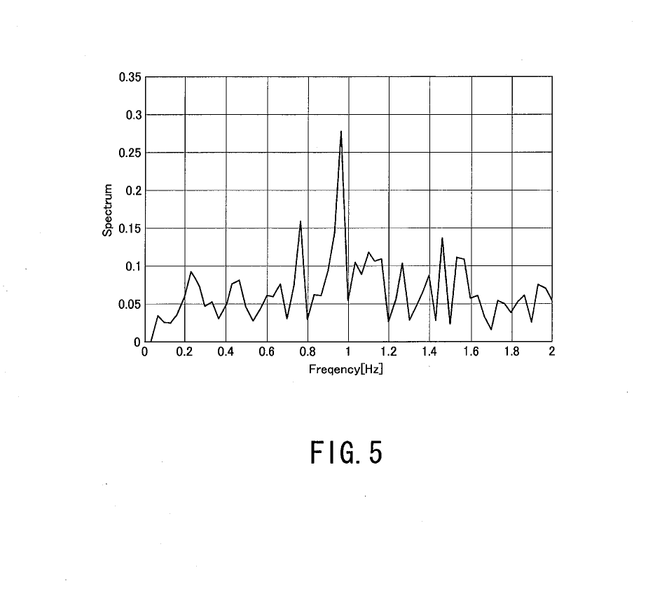

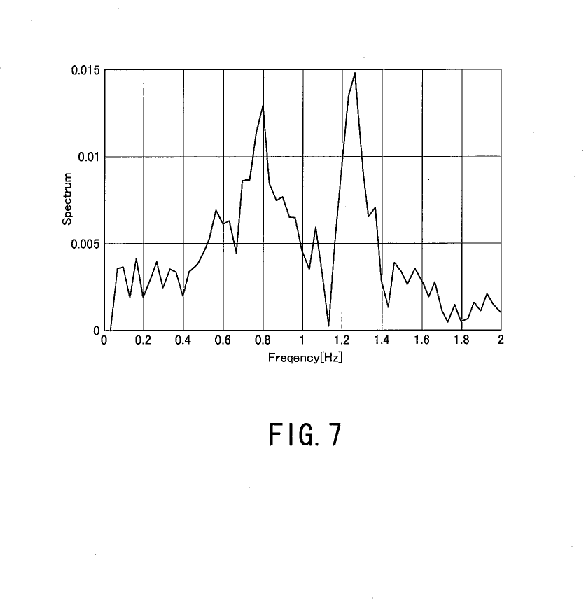

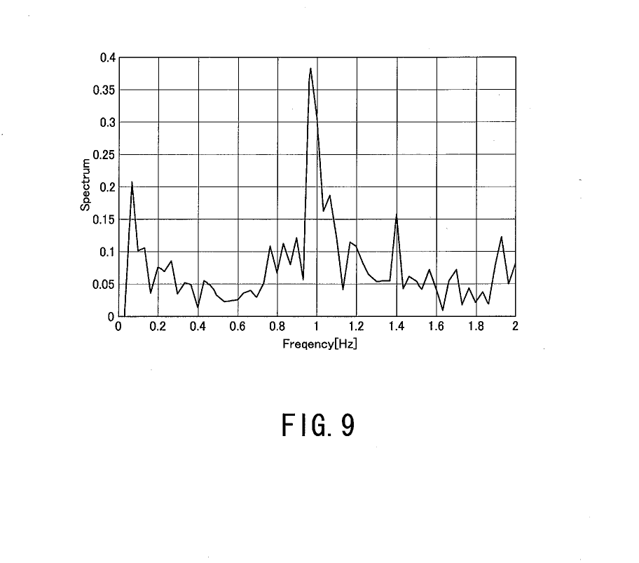

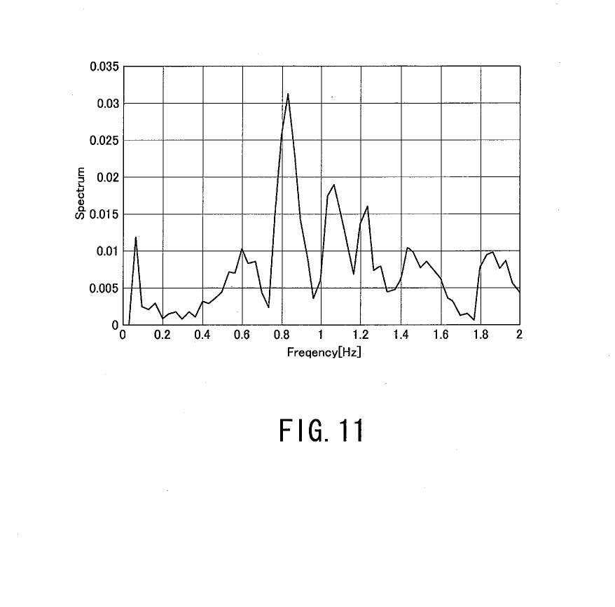

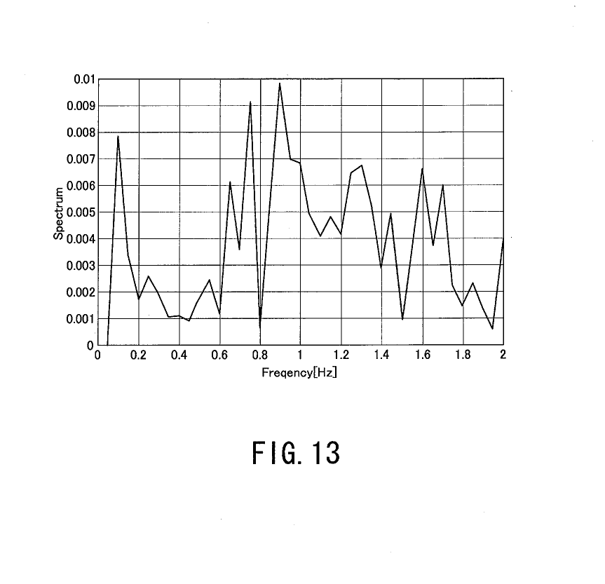

In addition, FIG. 5, FIG. 7, FIG. 9, FIG. 11 and FIG. 13 respectively show graphs in which the frequency power spectrums indicative of the occurrence frequencies of the peak components in the signals, as obtained through the frequency analysis of the phase difference signals in the respective states of Examples 1 to 5 as mentioned above, are plotted, wherein the abscissa of the graphs denotes a frequency [Hz], and the ordinate, a spectrum intensity [arb.u.].

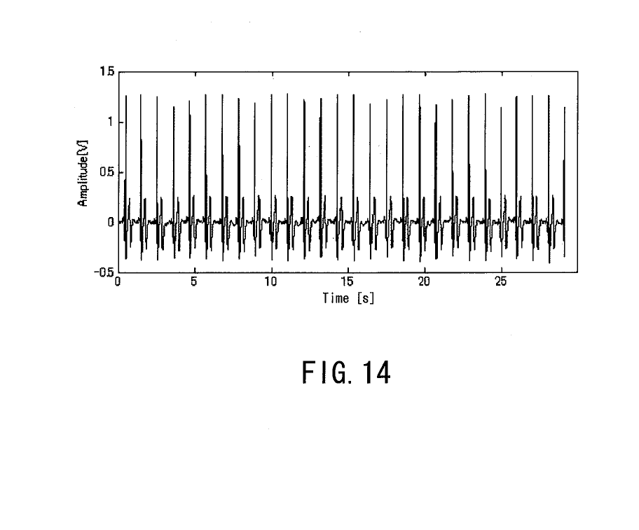

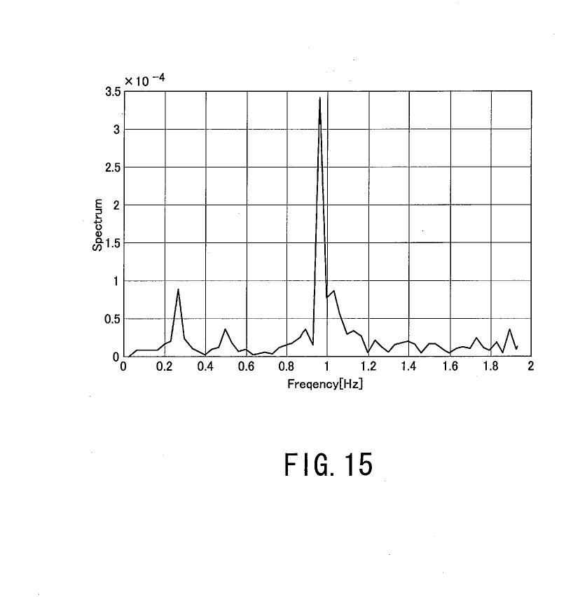

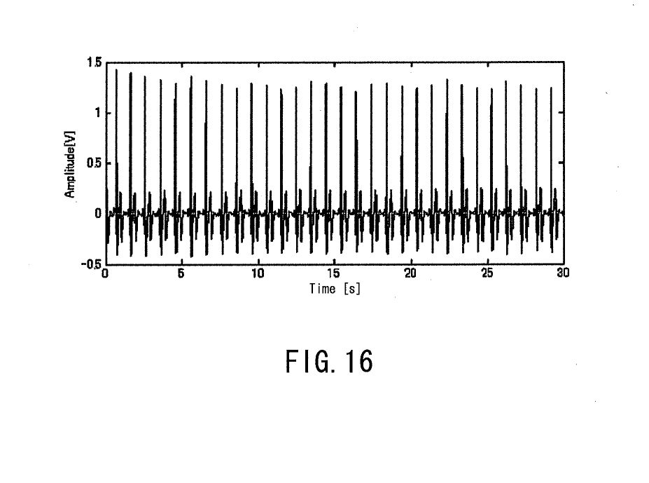

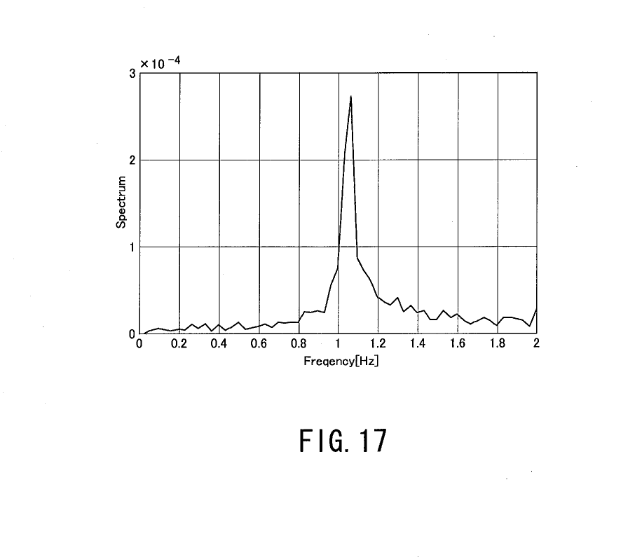

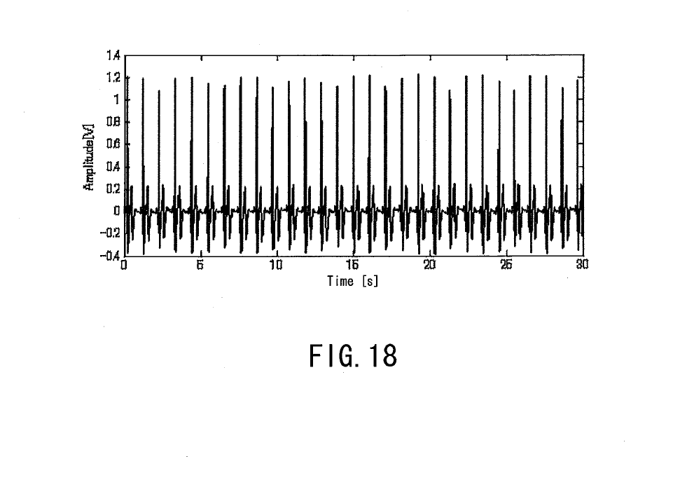

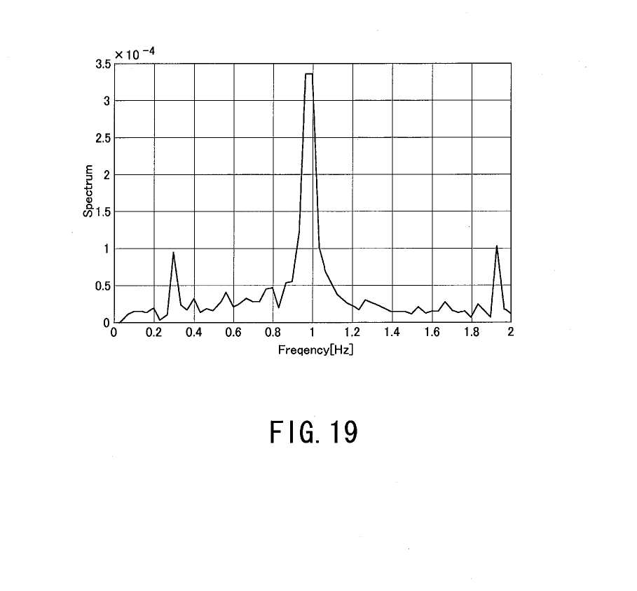

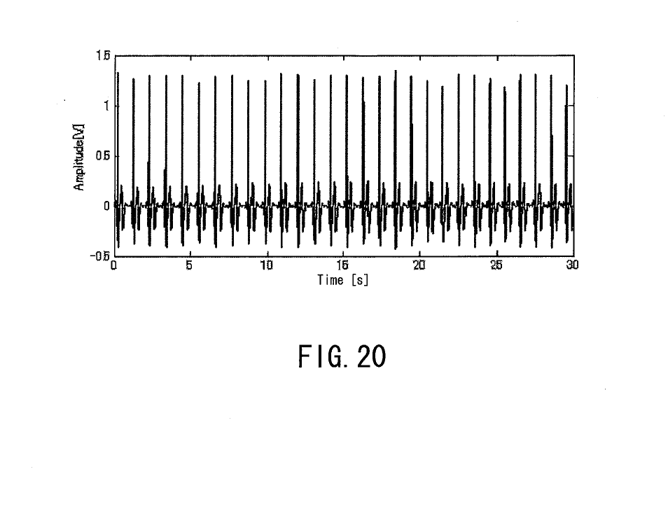

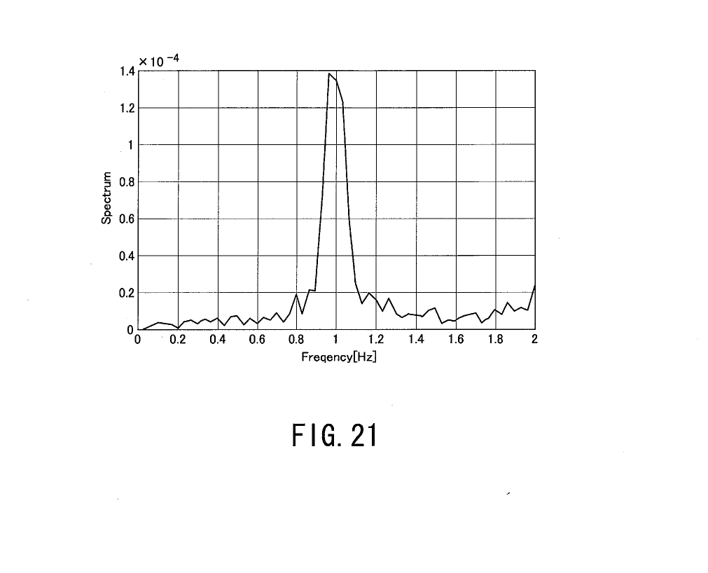

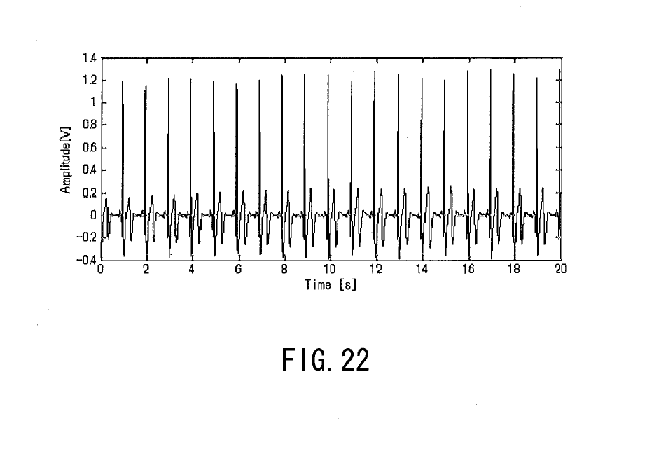

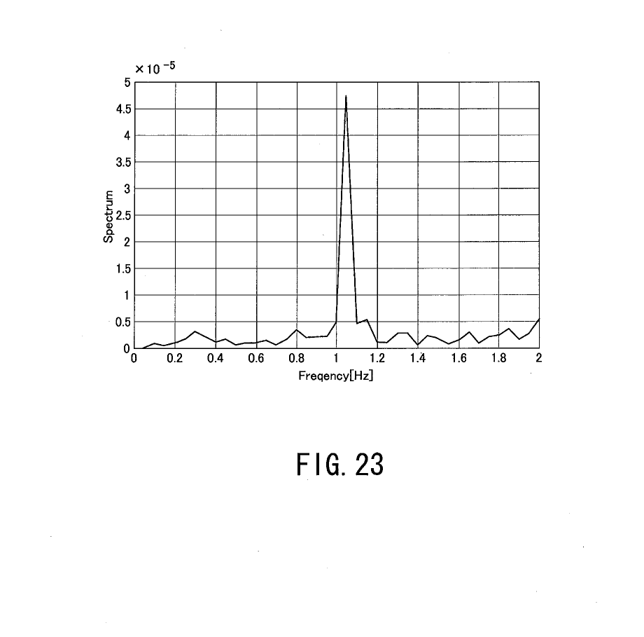

Further, FIG. 14, FIG. 16, FIG. 18, FIG. 20 and FIG. 22 respectively show graphs in which the signal waveforms in which the peaks of the heart-beats appear, as obtained the measurement of the heart-beat by the electrocardiograph in Comparative Examples 1 to 5, at the same time as the measurement in Examples 1 to 5 of the present invention as described above, are plotted, wherein the abscissa of the graphs denotes a lapse time [s], and the ordinate, amplitude [V]. FIG. 15, FIG. 17, FIG. 19, FIG. 21 and FIG. 23 respectively show graphs in which the frequency power spectrums as obtained through the frequency analysis of the signals in Comparative Examples 1 to 5, are plotted, wherein the abscissa of the graphs denotes a frequency [Hz], and the ordinate, a spectrum intensity [arb.u.], in a similar way. There was made a comparison between the frequency power spectrums as obtained for the phase difference signals and the power spectrums as obtained from the measurement results by the electrocardiograph in a similar way.

It was revealed that, although the phase difference signal obtained in the radiation state of the microwave in Example 1 of the present invention differed in signal level from the signal indicative of the heart-beat obtained by the electrocardiograph in Comparative Example 1 at the same time as Example 1 of the present invention, as shown in FIG. 4 and FIG. 14, the frequencies at the maximum peak positions of the frequency power spectrums as shown in FIG. 5 and FIG. 15 were substantially coincident with each other, and the results obtained by the use of the microwave and the results obtained by the electrocardiograph corresponded to each other with accuracy, the heart-beat frequency could be obtained by the use of the microwave, without causing any problem.

It was revealed that, although the phase difference signal obtained in the radiation state of the microwave in Example 3 of the present invention differed in signal level from the signal indicative of the heart-beat obtained by the electrocardiograph in Comparative Example 3 at the same time as Example 1 of the present invention, as shown in FIG. 8 and FIG. 18, the frequencies at the maximum peak positions of the frequency power spectrums as shown in FIG. 9 and FIG. 19 were substantially coincident with each other, and the results obtained by the use of the microwave and the results obtained by the electrocardiograph corresponded to each other with accuracy, the heart-beat frequency could be obtained by the use of the microwave, without causing any problem, in the similar manner.

It was revealed from the above that, when the antenna was directed to the human, the distance between the human and the antenna did not have any significant influence on the deriving of the heart-beat frequency.

To the contrary, the phase difference signal obtained in the radiation state of the microwave in Example 2 had a difference in level from the signal indicative of the heart-beat as obtained by the electrocardiograph at the same time as Example 2 of the present invention, as shown in FIG. 6 and FIG. 16, respectively, and had a smaller signal level also in comparison with the signal of Example 1 of the present invention in which the difference between the human within the radiation range and the antenna was set as the same condition. In addition, it was revealed that the frequency at the maximum peak position of the frequency power spectrum differed from that as obtained through the signal of the electrocardiograph, as shown in FIG. 7 and FIG. 17, respectively, and deviation of the radiation position inhibited a complete capture of the motion of the reflection surface on the body surface corresponding to the beat of heart, thus being difficult to obtain properly the heart-beat frequency. The same results were revealed in Example 4 of the present invention.

In addition, there was a large difference in level between the phase difference signal obtained in the radiation state of the microwave in Example 5 of the present invention, in which there was no reflected wave from the human, and the signal indicative of the heart-beat obtained by the electrocardiograph at the same time as Example 5 of the present invention, as shown in FIG. 12 and FIG. 22, respectively, and the signal level remarkably decreased also in comparison with the signals of each of the other examples. Further, the spectrum intensity of the frequency power spectrum was lower than each of the examples, as shown in FIG. 13 and FIG. 23, respectively, and the maximum peak position did not clearly appear unlike that obtained through the signal of the electrocardiograph or in the other examples. It was revealed that the phase difference signal as obtained and the frequency power spectrum in case of the non-existence of the human in the radiation range was much different from that in case of the existence of the human in the radiation range.

It could be confirmed in this manner that, in relation to the process of radiating the microwave, while changing the radiation state in the region of the object to be measured, and receiving the reflected wave, and determining the frequency power spectrum from the phase difference signal, the phase difference signal, etc. as obtained in case of the existence of the human in the radiation region was quite different from that as obtained in case of the non-existence of the human in the radiation region. It is apparent from these facts that setting threshold values for the signal level, the spectrum shape, etc., to make a determination permits to recognize the existence of the human. It is also apparent that the scanning operation is performed along with the radiating and receiving operation of the microwave, and the radiation position at a certain time during the scanning operation is associated with the phase difference signal as obtained, thus making it possible to detect the existing position of the human in the region.

It was also be confirmed that, when the antenna captured a position on the body surface in which a micro-motion corresponding to the beat of the heart of the human occurred, in case of the human entering the radiation range of the antenna, the heart-beat frequency of the human could be obtained even in a non-contact state with the similar accuracy to the measurement by the electrocardiograph. There is fully achievable an application of recognizing, from such a heart-beat frequency, a plurality of humans as detected as existing in the region, or making a stress assessment by determining the HRV, etc., to recognize the condition of the human.

### REFERENCE SIGNS LIST

2 living organism information detection system
20 electromagnetic wave transmitting and receiving unit
21 transmitting and receiving section
21a radiating antenna
21b microwave oscillator
21c receiving antenna
21d directional coupler
21e high-frequency oscillator
21f distributor
21g up converter
21h mixer section
21i antenna driving mechanism
21j antenna controlling section
22 adjusting unit
23 quadrature detecting unit
24 calculating unit
25 signal analyzing unit
26 living organism information processing unit
27 recording unit
28 cross-checking unit
50 region of an object to be measured
60 massager
70 human

What is claimed is:

1. A living organism information detection system, comprising:
an electromagnetic wave transmitting and receiving unit that radiates an electromagnetic wave having a predetermined continuous frequency with a predetermined narrow directivity to a region of an object to be measured, and receives a reflected wave, and outputs a phase difference signal between an radiated wave and the reflected wave at respective positions of a whole of said region by an execution with a scanning of radiation and receipt; and
a signal analyzing unit that analyzes the phase difference signal in correspondence with respective radiated positions as scanned in said region of the object to be measured, and, in case where there is a temporal variation in the phase difference signal, detects, one or a plurality of predetermined positions corresponding to a range of a time direction of the signal in which said variation has occurred, as an existing position of a living organism, and detects the temporal variation in the phase difference signal corresponding to said existing position, as a living organism information indicative of a condition of the living organism existing in the existing position.

2. The living organism information detection system, as claimed in claim 1, comprising:
a living organism information processing unit that obtains a peak component of a signal, which is generated substantially periodically in response to a roughly steady micro-motion representing vital signs of a human as the living organism, from the temporal variation in the phase difference signal as the living organism information as detected by said signal analyzing unit, to determine an appearance interval information of said vital signs,
wherein:
said electromagnetic wave transmitting and receiving unit further radiates the electromagnetic wave to the one or the plurality of existing positions corresponding to said living organism information as detected by said signal analyzing unit, while continuing the scanning, and maintains a state in which the reflected wave is to be received, for a predetermined period of time until the phase difference signal having a signal length necessary for determination of the appearance interval information may be obtained by said living organism information processing unit, and outputs newly a phase difference signal,
said signal analyzing unit detects the temporal variation in the phase difference signal as the living organism information for the respective existing positions, from said phase difference signal as newly outputted, and
said living organism information processing unit determines the appearance interval information of said vital signs for the respective living organism, from the temporal variation in the phase difference signal for the respective existing positions.

3. The living organism information detection system, as claimed in claim 2, comprising:
a recording unit that records as a database for the respective living organism, the appearance interval information of said vital signs as obtained by said living organism information processing unit, together with the existing position; and
a cross-checking unit that makes a cross-checking between the living organism information as recorded by said recording unit and the appearance interval information of said vital signs for the respective living organism as newly obtained by said living organism information processing unit to identify the living organism.

4. The living organism information detection system, as claimed in claim 1, wherein:
the scanning in said electromagnetic wave transmitting and receiving unit is achieved through radiation by a phase control of an array antenna or deflection of a receiving direction.

5. The living organism information detection system, as claimed in claim 2, wherein:
the scanning in said electromagnetic wave transmitting and receiving unit is achieved through radiation by a phase control of an array antenna or deflection of a receiving direction.

6. The living organism information detection system, as claimed in claim 3, wherein:
the scanning in said electromagnetic wave transmitting and receiving unit is achieved through radiation by a phase control of an array antenna or deflection of a receiving direction.
